(19) United States
(12) Patent Application Publication
(10) Pub. No.: US 2026/0124165 A1
(43) Pub. Date: May 7, 2026

(54) COMPOSITIONS COMPRISING CREATINE FOR USE IN TELOMERE LENGTHENING

(71) Applicant: Sergej Ostojic, Pancevo (RS)

(72) Inventor: Sergej Ostojic, Pancevo (RS)

(21) Appl. No.: 18/934,264

(22) Filed: Nov. 1, 2024

(51) Int. Cl. A61K 31/198; A61P 43/00

(52) U.S. Cl. CPC A61K 31/198; A61P 43/00

(57) ABSTRACT

The present invention pertains to a pharmaceutical composition comprising creatine or its physiologically acceptable derivatives, precursors, salts, or adducts for facilitating telomere length. Another aspect of the invention concerns the utilization of a composition containing creatine or its derivatives, precursors, salts, or adducts as a dietary supplement to extend telomere length in aged subjects and smokers.

### REFERENCES CITED

### U.S. Patent Documents

11007210 B2 5/2021 Ramunas et al.

### Other Publications

Astuti Y, Wardhana A, Watkins J, Wulaningsih W; PILAR Research Network. Cigarette smoking and telomere length: A systematic review of 84 studies and meta-analysis. Environ Res. 2017 Oct;158:480-489. doi: 10.1016/j.envres.2017.06.038.

Balestrino M, Adriano E. Beyond sports: Efficacy and safety of creatine supplementation in pathological or paraphysiological conditions of brain and muscle. Med Res Rev. 2019 Nov;39(6): 2427-2459. doi: 10.1002/med.21590.

Brosnan M E, Brosnan J T. The role of dietary creatine. Amino Acids. 2016 Aug;48(8):1785-91. doi: 10.1007/s00726-016-2188-1.

Candow D G, Chilibeck P D, Forbes S C, Fairman C M, Gualano B, Roschel H. Creatine supplementation for older adults: Focus on sarcopenia, osteoporosis, frailty and Cachexia. Bone. 2022 Sep;162:116467. doi: 10.1016/j. bone.2022.116467.

Forbes S C, Cordingley D M, Cornish S M, Gualano B, Roschel H, Ostojic S M, Rawson E S, Roy B D, Prokopidis K, Giannos P, Candow D G. Effects of Creatine Supplementation on Brain Function and Health. Nutrients. 2022 February 22;14(5):921. doi: 10.3390/nu14050921.

Galié S, Canudas S, Muralidharan J, García-Gavilán J, Bulló M, Salas-Salvadó J. Impact of Nutrition on Telomere Health: Systematic Review of Observational Cohort Studies and Randomized Clinical Trials. Adv Nutr. 2020 May 1;11(3):576-601. doi: 10.1093/advances/nmz107.

Gao X, Yu X, Zhang C, Wang Y, Sun Y, Sun H, Zhang H, Shi Y, He X. Telomeres and Mitochondrial Metabolism: Implications for Cellular Senescence and Age-related Diseases. Stem Cell Rev Rep. 2022 Oct;18(7):2315-2327. doi: 10.1007/s12015-022-10370-8. Epub 2022 April 23.

Herrmann M, Pusceddu I, März W, Herrmann W. Telomere biology and age-related diseases. Clin Chem Lab Med. 2018 July 26;56(8):1210-1222. doi: 10.1515/cclm-2017-0870.

Institute of Medicine. 2006. Dietary Reference Intakes: The Essential Guide to Nutrient Requirements. Washington, D C: The National Academies Press. https://doi.org/10.17226/11537.

Jaskelioff M, Muller F L, Paik J H, Thomas E, Jiang S, Adams A C, Sahin E, Kost-Alimova M, Protopopov A, Cadiñanos J, Horner J W, Maratos-Flier E, Depinho R A. Telomerase reactivation reverses tissue degeneration in aged telomerase-deficient mice. Nature. 2011 January 6;469(7328):102-6. doi: 10.1038/nature09603.

Kreider R B, Stout J R. Creatine in Health and Disease. Nutrients. 2021 Jan. 29;13(2):447. doi: 10.3390/nu13020447.

McCall W, Persky A M. Pharmacokinetics of creatine. Subcell Biochem. 2007;46:261-73.

Ostojic S M, Ostojic J, Drid P, Vranes M, Jovanov P. Dietary guanidinoacetic acid increases brain creatine levels in healthy men. Nutrition. 2017 Jan;33:149-156. doi: 10.1016/j.nut.2016.06.001.

Ostojic S M, Forbes S C. Perspective: Creatine, a Conditionally Essential Nutrient: Building the Case. Adv Nutr. 2022 Feb. 1;13(1):34-37. doi: 10.1093/advances/nmab111.

Ostojic S M. Creatine as a food supplement for the general population. Journal of Functional Foods. 2021 Aug. 1;83:104568.

Ostojic S M. Low Tissue Creatine: A Therapeutic Target in Clinical Nutrition. Nutrients. 2022 Mar. 15;14(6):1230. doi: 10.3390/nu14061230.

Wang Q, Zhan Y, Pedersen NL, Fang F, Hägg S. Telomere Length and All-Cause Mortality: A Meta-analysis. Ageing Res Rev. 2018 Dec;48:11-20. doi: 10.1016/j.arr.2018.09.002.

### FIELD OF THE INVENTION

The current invention pertains to pharmaceutical compositions containing creatine and its derivatives useful in the extension of telomeres in a subject in need thereof. Such subjects include those with shortened telomeres as a consequence of aging, stress exposure, chronic inflammation, or other illnesses. Another objective of the invention involves employing creatine and creatine derivatives as food supplements to prepare a diet aimed at lengthening telomeres.

### DESCRIPTION OF THE PRIOR ART

Telomeres are repetitive sequences of DNA found at the ends of chromosomes. They function as protective caps, preserving the integrity of the genetic material during cell division and preventing the loss of genetic information. As cells divide and replicate, their telomeres gradually shorten, acting as a biological clock that tracks cellular aging and replication limit. Maintaining adequate telomere length is essential for healthy cell function and longevity.

Telomere shortening has been shown to accompany physical disease states that are associated with aging and stress exposure, including diabetes mellitus, obesity, heart disease, chronic obstructive pulmonary disease (COPD), asthma, as well as psychiatric illnesses, such as depression, anxiety, post-traumatic stress disorder (PTSD), bipolar disorder, and schizophrenia. Shorter telomeres have been associated with increased incidence of diseases and poor survival.

Telomere length typically shortens with age, demonstrated by an average annual reduction of approximately 30-35 base pairs, as found in a study by Herrmann, M. et al (Clin. Chem. Lab. Med. 2018, 56, 1210-1222). Telomere length was found to be 11% shorter in individuals who were identified as ever smokers compared to those who were identified as never smokers, as reported in a study by Astuti, Y. et al. (Environ. Res. 2017, 158, 480-489). Individuals with the shortest telomere length had 44% higher risk of all-cause mortality than those with the longest telomere length, as found in a study by Wang, M. et al. (Ageing Res. Rev. 2018, 48, 11-20).

People with shorter telomeres often experience accelerated aging, age-related decline in function or appearance, increased risk of age-related diseases, immune system decline, stress sensitivity, reduced regenerative capacity, risk of mental health conditions and reduced longevity. These symptoms frequently endure for extended periods, often spanning several months, in numerous instances. Recommendations for these subjects are usually limited to behavioral advice, such as to consume healthy diet, manage chronic stress, and avoid smoking, alcohol consumption and exposure to environmental toxins. While lifestyle factors may influence telomere length, individual responses can vary. Genetics also play a significant role in determining telomere length.

Telomeres shorten over time due to oxidative damage, and decreased energy production in the cell (Stem Cell Rev. Rep. 2022, 18, 2315-2327). Telomere shortening contributes to organ dysfunction, deterioration of physical prowess, a decline in youthful appearance, loss of mental acuity, and susceptibility to diseases. Conversely, increasing telomere length in subjects with short telomeres reverses tissue degeneration and eliminates degenerative phenotypes across multiple organs (Nature. 2011, 469, 102-106).

Several compounds and compositions have been identified to be useful in the extension of telomeres (US 11007210 B2, Ramunas et al. 2021). The compounds comprise a synthetic ribonucleic acid. These compositions are targeted to increase telomerase activity, an enzyme responsible for maintenance of the length of telomeres by addition of guanine-rich repetitive sequences. These methods risk genomic modification and may cause unwanted side effects.

Adequate nutrition may potentially help mitigating the shortening of telomeres. Consuming a balanced diet rich in fruits, vegetables, whole grains, complete proteins, and healthy fats may support overall health and potentially affect telomere length. Several micronutrients have been associated with telomere length maintenance, including vitamins D, E and C, omega-3 fatty acids, zinc and magnesium. Impact of nutrition on telomere length is described in Galiè, S. et al. (Adv. Nutr. 2020, 11, 576-601).

Creatine is a naturally occurring nitrogenous organic acid found in vertebrates. It's primarily synthesized in the liver, kidneys, and pancreas and is also obtained through diet, primarily from animal products like meat and fish. In the body, creatine is converted into phosphocreatine, which serves as a rapidly mobilizable reserve of high-energy phosphates used in adenosine triphosphate (ATP) regeneration. ATP is a molecule that stores and transports energy within cells, crucial for various biological processes (Brosnan, M. E. et al., Amino Acids 2016, 48, 1785-1791; Ostojic, S. M., Forbes, S. C. Adv. Nutr. 2022, 13, 34-37).

Supplemental creatine is commonly used to enhance work performance, as it increases the phosphocreatine stores in muscles, allowing for more rapid ATP production. Beyond its role in energy metabolism, creatine and creatine derivatives have also been investigated for potential benefits in brain health, aging, and certain medical conditions (McCall, W., Persky, A M. Subcell. Biochem. 2007, 46, 261-273; Balestrino, M., Adriano, E. Med. Res. Rev. 2019, 39, 2427-2459; Kreider, R. B., Stout, J. R. Nutrients 2021, 13, 447; Candow, D. G. et al. Bone. 2022, 162, 116467; Forbes, S. C. et al. Nutrients. 2022, 14, 921). Creatine or creatine analogs appear to partially or totally restore impaired cell bioenergetics and attenuate clinical features of many maladies (Ostojic, S. M. Nutrients. 2022, 14, 1230)

The problem to be solved by the present invention is to enhance the telomere length in individuals who could potentially benefit from telomere extension, such as subjects afflicted by, or susceptible to, age-related or other ailments.

### DESCRIPTION OF THE INVENTION

The problem is solved by administration of creatine in subjects in need thereof. Creatine facilitates the restoration of telomere length that has been depleted due to aging and/or other conditions.

The administration of creatine is especially useful for subjects in middle age, particularly those who experience environmental stress such as smoking. Creatine utilization significantly improved telomere length in adults over 50 years or age compared to the application of placebo.

Accordingly, a first embodiment of the present invention pertains to a pharmaceutical composition that contains creatine or its physiologically acceptable derivatives, salts, precursors or adducts, intended for application in telomere lengthening.

A second embodiment of this invention involves employing creatine or its physiologically acceptable derivatives, salts, precursors or adducts as a dietary supplement or for preparing a dietary regimen facilitating the restoration of telomere length, subsequent to aging and/or environmental toxins.

Through the administration of creatine, telomere elongation was accompanied by a notable elevation in creatine concentration within the brain, while telomere length and creatine levels remained unresponsive to placebo administration. The administration of creatine facilitates the enhancement of tissue creatine levels, thereby providing support for telomere length maintenance.

The pharmaceutical composition stated in the first embodiment and the composition designated as a dietary supplement in the second embodiment shall henceforth be denoted as a creatine composition, creatine-containing composition, or composition encompassing creatine. These terms, including “creatine composition,” “creatine-containing composition,” or “composition comprising creatine,” also encompass physiologically acceptable creatine derivatives, creatine precursors, creatine salts, and/or creatine adducts unless specifically indicated otherwise.

The creatine-containing compositions within this invention are notably advantageous to support telomere length. Telomere shortening is characterized by reduced regenerative capacity. Telomere shortening can happen as a consequence of aging and several acute and chronic conditions. However, telomere shortening also occurs after exposure to environmental toxins, in particular tobacco smoke. Telomere shortening often persists for months in smokers.

Telomere shortening within the meaning of the invention comprises particularly leukocyte telomere shortening caused by aging and environmental stress.

Telomere length due to aging can be surprisingly improved by administration of creatine. Creatine, also recognized as alpha-Methylguanidioacetic acid, is a natural substance found in the bodies of animals and humans. It's also referred to by alternative names such as N-amidinosarcosine, N-methyl-N-guanylglycine, N-carbamimidoyl-N-methylglycine or N-(aminoiminiomethyl)-N-methylglycine. Creatine is obtained from animal-derived foods or is available in greater concentrations as a dietary supplement.

In addition to creatine, the invention allows for the use of physiologically acceptable creatine derivatives. These derivatives may encompass naturally occurring compounds like phosphocreatine, or creatine precursors capable of releasing creatine under physiological conditions, such as guanidinoacetic acid. Preferably, physiologically acceptable creatine derivatives include but not limited to creatine hydrates, creatine esters, phosphocreatine, guanidinoacetic acid, creatinol-O-phospate or a combination thereof.

Preferred creatine salts, creatine adducts, salts of physiologically acceptable creatine derivatives, and adducts of these derivatives ideally encompass acetates, aconitates, aminobutyrates, ascorbates, aspartates, chlorides, citrates, decanoates, fumarates, gluconates, hemi-sulfates, hydrochlorides isocitrates, ketoglutarates, ketoisocaproate, malates, maleates, nitrates, orotate, oxalates, oxaloacetates, phosphates, pyruvates, ribosides, succinates, sulfates, tartrates, taurinates, tetrahydrates, sodium, potassium, calcium, magnesium salts, guanidinoacetic acid adducts, or a combination thereof.

Commencement of creatine treatment may be initiated during conditions inducing telomere shortening, preferably within approximately four weeks after the ailment. Additionally, the administration of creatine is ideally initiated at age 50 years to facilitate telomere length. Nonetheless, the administration of creatine even after the initiation of telomere shortening remains feasible.

The typical duration of creatine supplementation ranges from 6 months to 18 months or more, with a preference for a duration between 9 and 12 months, contingent upon the features exhibited by the subject requiring treatment.

The recommended dosage of creatine for administration falls within the range of 1 gram to 4 grams per day. Ideally, the dosage should range between 2 grams to 3 grams. These dosages are comparable to the typical recommendations intended for the general population (Ostojic, S M. J. Funct. Foods, 2021, 83, 104568). The dosage of creatine in the composition may be varied depending on e.g. age, weight, gender, condition, disease and course of treatment of an individual subject in need thereof.

The daily administration of the creatine dose can occur once a day, ideally during breakfast, or divided into two doses taken during specific meal times, such as breakfast and dinner, allowing for flexibility in optimizing intake for enhanced efficacy and convenience.

If the provided creatine composition encompasses a creatine derivative, precursor, adduct, or salt, the quantity of the creatine element contained in it determines the daily dosage within the previously specified ranges.

The creatine composition mentioned in this description is preferably administered orally, in the form of granules, powders, tablets or capsules.

Granules or powders comprising the creatine composition are utilized as aqueous suspensions or in water-soluble forms. Water-soluble granules or powders are generally preferred due to low solubility of creatine and creatine derivatives. Enhancing the water solubility of creatine or its derivatives can be achieved by using water-soluble salts or adducts. To augment water solubility, the employment of acids or complexing agents is beneficial in producing corresponding creatine salts or creatine derivative salts. Suitable acids and complexing agents are selected from the group acetic acid, aminobutyric acid, ascorbic acid, aspartic acid, citric acid, fumaric acid, gluconic acid, hydrochloric acid, isocitric acid, a-ketoglutaric acid, a-ketoisocaproic acid, malic acid, maleic acid, orotic acid, oxalic acid, oxaloacetic acid, pyruvic acid, ribonic acid, succinic acid, and tartaric acid. Amino acids can also be employed to enhance the solubility of creatine and its derivatives, including glycine, serine, threonine, cysteine, glutamate, and asparagine. In addition, sodium, potassium, calcium, and magnesium salts may be utilized to bolster the water solubility of creatine or its derivatives. The molar ratio between creatine or its derivatives and the specified acids or complexing agents typically ranges from 3:1 to 1:3.

The granules and powders can also serve as constituents for formulating food products that facilitates telomere length, including beverages, breakfast cereals, candy, condiments, confectionery, dairy, dips and spreads, energy bars, flours, frozen desserts, and meat substitutes.

The creatine composition can be formulated into tablets, either in its pure form or combined with excipients. These excipients are inactive pharmacological ingredients, including binders, fillers, antioxidants, preservatives, stabilizers, anti-caking agents, lubricants, disintegrants, flavors, pigments, and similar additives.

A diverse array of compounds may be utilized as binders or fillers in the formulation, such as cellulose derivatives like hydroxypropyl cellulose, or hydroxypropyl methylcellulose, starches, polyvinylpyrrolidone, and gelatin; lactose, microcrystalline cellulose, calcium phosphate, and mannitol. Typical antioxidants may be selected from a group of vitamin C, sodium metabisulfite, butylated hydroxytoluene, butylated hydroxyanisole and vitamin E. Suitable preservatives are benzoic acid and its salts, including sodium benzoate; sorbic acid and its salts, including potassium sorbate; and parabens, like methylparaben or propylparaben.

Typical anti-caking agents may be selected from a group of magnesium stearate, silicon dioxide and talc. Examples of lubricants include stearic acid and polyethylene glycol. Common types of stabilizers and disintegrants are cellulose derivatives, including microcrystalline cellulose, povidone, croscarmellose sodium or crospovidone, sodium starch glycolate, polyvinyl alcohol, polyethylene glycol, tocopherols, and cross-linked polyvinylpyrrolidone. Common natural or synthetic flavors, including fruit flavors, mint flavors, vanilla flavors, chocolate or cocoa flavors, citrus flavors and berry flavors can be used as flavors. Some typical pigments used in the formulations include: iron oxides, titanium dioxide, indigo carmine, tartrazine, sunset yellow, allure red and brilliant blue.

Particularly useful coatings for tablets related to the invention disclosed herein are film coatings, including hydroxypropyl methylcellulose, polyvinyl alcohol, and polyethylene glycol coatings; enteric coatings, including cellulose acetate phthalate, hydroxypropyl methylcellulose phthalate, and methacrylic acid copolymers; functional coatings, including ethyl cellulose or acrylic resins; and barrier coatings, including shellac or other polymers. Capsules are typically coated with gelatin for encapsulation purposes.

Preferred tablets comprise at least: (1) 20-100 weight percentage creatine or its physiologically acceptable derivatives, salts, precursors or adducts thereof; (2) 0 -10 weight percentage antioxidants, particularly vitamin C; (3) 0-10 weight percentage preservatives, particularly potassium sorbate; (4) 0-20 weight percentage anti-caking agent, particularly magnesium stearate; (5) 0 -10 weight percentage lubricants, preferably stearic acid; (6) 0-10 weight percentage stabilizers, preferably microcrystalline cellulose; (7) 0-10 weight percentage flavors, preferably mint flavor.

Preferred capsules comprise at least: (1) 20-90 weight percentage creatine or its physiologically acceptable derivatives, salts, precursors or adducts thereof; (2) 0 -10 weight percentage antioxidants, particularly vitamin C; (3) 0-10 weight percentage preservatives, particularly potassium sorbate; (4) 0-20 weight percentage anti-caking agent, particularly magnesium stearate; (5) 0 -10 weight percentage lubricants, preferably stearic acid; (6) 0-10 weight percentage stabilizers, preferably microcrystalline cellulose; (7) 0-10 weight percentage flavors, preferably mint flavor; (8) 10-90 weight percentage gelatin.

The aforementioned components are applicable not solely in tablets but also in granules or powders containing creatine or its physiologically acceptable derivatives, salts, precursors or adducts. Specifically, binders, fillers, antioxidants, preservatives, stabilizers, anti-caking agents, lubricants, disintegrants, flavors, pigments, and similar additives are integral constituents of the creatine composition in alignment with the current invention.

The granules or powders as outlined in the present invention typically encompass 10 to 100 weight percentage of creatine or its physiologically acceptable derivatives, salts, precursors or adducts.

Additional combinations involving creatine, creatine derivatives, salts, precursors, or adducts thereof in conjunction with telomere-lengthening compounds can be advantageous. Preferred telomere-lengthening agents include telomerase activation simulators, such as cycloastragenol, resveratrol, AGS-499, TA-65, GRN510; synthetic sex hormones, such as danazol; growth factors, such as IGF-1, FGF-2, VEGF; erythropoietin; estrogen; and leptin.

Creatine, creatine derivatives, or their precursors/salts/adducts are preferably employed in conjunction with a telomere-facilitating diet as a preferred embodiment of the present invention. This recommended diet aims to supply all the necessary nutrients for optimal telomere viability, including vitamins, minerals, essential fatty acids, essential and conditionally essential amino acids, complex carbohydrates. Sufficient amounts are particularly required for B vitamins (thiamin, riboflavin, niacin, pantothenic acid, pyridoxine, biotin, folic acid, cobalamin), vitamin C, vitamin E, vitamin A and beta-carotene, vitamin K, choline, calcium, chromium, copper, fluoride, iodine, iron, magnesium, manganese, molybdenum, phosphorus, potassium, selenium, sodium, chloride, sulfate, zinc, arsenic, boron, nickel, selenium and vanadium. Nutrient requirements are issued by the Institute of Medicine (Dietary Reference Intakes: The Essential Guide to Nutrient Requirements. Washington, D C: The National Academies Press., 2006)

Further nutritional compounds useful in telomere-facilitating diet include phytonutrients with antioxidant properties, such as ployphenols, carotenoids, flavonoids, terpenoids, alkaloids and organosulfur compounds; non-essential amino acids, including alanine, arginine, asparagine, aspartic acid, cysteine, glutamic acid, glutamine, glycine, proline, serine, and tyrosine; enzymes, including amylase, protease, glucose amylase, lipase, cellulase, lactase, pectinase; alpha-lipoic acid; coenzyme Q10; nicotinamide adenine dinucleotide; para-aminobenzoic acid and gamma-amino butyric acid; inositol, taurine, carnosine, carnitine, anserine, and 4-hydroxyproline.

The optimal nutrients for a telomere-facilitating diet can be supplied by choosing appropriate foods or by incorporating the necessary constituents through nutritional supplements.

### BRIEF DESCRIPTION OF FIGURES

FIG. 1. Changes in telomere length (presented as telomere length relative to standard reference DNA, T/S ratio) after 12-month supplementation with 2.5 grams of creatine (CR) per day or placebo in adults aged 50 years. Left pair of bars representing the group of patients receiving placebo, right pair of bars representing the group of patients receiving creatine. Grey bars representing telomere length at baseline, black bars representing telomere length after 12 months of intervention. Error bars indicate standard error. Asterisk (*) indicates significant difference within-group at P<0.05. Double dagger (‡) indicates statistical significance for 2-way ANOVA interaction effect (time vs. treatment).

FIG. 2. Changes in telomere length (presented as telomere length relative to standard reference DNA, T/S ratio) after 12-month supplementation with 1.5 grams of creatine and 1.5 grams of guanidinoacetic acid per day or placebo in smokers aged 30-50 years. Left pair of bars representing the group of patients receiving placebo, right pair of bars representing the group of patients receiving creatine and guanidinoacetic acid. Grey bars representing telomere length at baseline, black bars representing telomere length after 12 months of intervention. Error bars indicate standard error. Asterisk (*) indicates significant difference within-group at P<0.05. Double dagger (‡) indicates statistical significance for 2-way ANOVA interaction effect (time vs. treatment).

FIG. 3. Changes in creatine levels in brain frontal grey matter after 12-month supplementation with 1.5 grams of creatine and 1.5 grams of guanidinoacetic acid per day (CR+GAA) or placebo in smokers aged 30-50 years. Left pair of bars representing the group of patients receiving placebo, right pair of bars representing the group of patients receiving creatine and guanidinoacetic acid. Grey bars representing creatine levels in brain frontal grey matter at baseline, black bars representing creatine levels in brain frontal grey matter after 12 months of intervention. Error bars indicate standard error. Asterisk (*) indicates significant difference within-group at P<0.05. Double dagger (‡) indicates statistical significance for 2-way ANOVA interaction effect (time vs. treatment).

FIG. 4. Changes in telomere length (presented as telomere length relative to standard reference DNA) after 12-month supplementation with 2.5 grams of creatine per day or placebo in adults aged 65 years and over. Grey bar representing telomere length at 12-month follow-up in the placebo group, black bars representing telomere length at 12-month follow-up in the creatine group. Error bars indicate standard error. Asterisk (*) indicates significant difference within-group at P<0.05.

### WORKING EXAMPLES

### Example 1: Creatine for Telomere Length in 50-year Old Adults

A study was conducted to assess the impact of creatine supplementation on telomere length in 94 men and women aged 50 years (age 50.2±0.6 years; 61 females). This investigation followed a randomized controlled pretest-posttest control group experimental design. Eligible participants for the trial met specific criteria, including an age of 50 years, absence of any acute or chronic diseases, and no prior history of regular creatine supplementation.

The experimental group received a daily dose of 2.5 grams of creatine, while the control group received an equivalent quantity of dietary fiber in the form of a placebo. Participants were instructed to consume the intervention once daily during breakfast, blending the experimental powder (creatine) or control powder (placebo) with 250 mL of lukewarm water and consuming it immediately afterward. Both interventions shared similar appearance, texture, and sensory characteristics.

The intervention period lasted 12 months, during which participants were advised to refrain from using any other dietary supplements. Telomere length was assessed at baseline and at the 12-month follow-up using the quantitative polymerase chain reaction method to measure telomere length relative to standard reference DNA, following detailed procedures described elsewhere (Cawthon, R. M., Nucleic Acids Res, 2002, 30, e47).

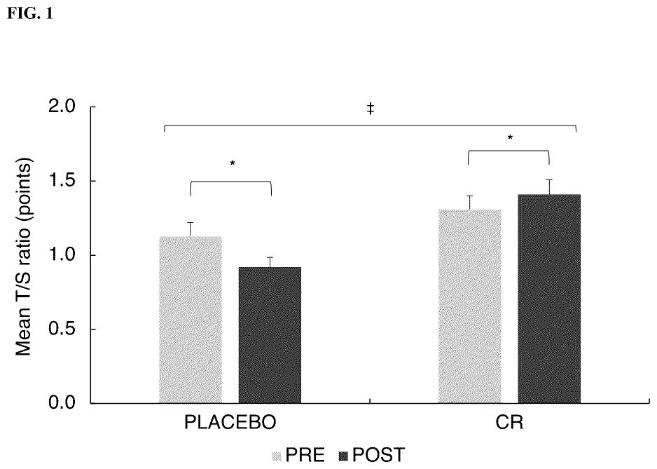

At the 12-month mark, the total number of participants randomized, who received the intended treatment, and were analyzed for the primary outcome was 94, comprising 45 in the experimental group and 49 in the control group. Changes in telomere length after intervention with 2.5 grams of creatine per day for 12 months compared to telomere length of the placebo group are depicted in FIG. 1.

The participants receiving 2.5 grams of creatine per day experienced an increase in telomere length at 12-month follow-up as compared to the baseline (pre-intervention) levels (P<0.05). The participants receiving placebo experienced a significant decrease in telomere length at 12-month follow-up as compared to the baseline (pre-intervention) levels (P<0.05). Two-way ANOVA with repeated measures revealed a significant difference (treatment vs. time interaction) between interventions in telomere length (P<0.05), with the creatine group superior to placebo to extend telomere length. No participants reported any side effects of creatine or placebo.

### Example 2: Creatine and Guanidinoacetic Acid for Telomere Length and Brain Creatine in Smokers

A study was conducted to assess the impact of creatine supplementation on telomere length and brain creatine levels in sixteen male and female smokers aged 30-50 years (age 42.5±12 years; 8 females). This investigation followed a randomized controlled pretest-posttest control group experimental design. Eligible participants for the trial met specific criteria, including current smoking, age of 30-50 years, absence of any acute or chronic diseases, and no prior history of regular creatine supplementation.

The experimental group received a daily dose of 1.5 grams of creatine and 1.5 grams of guanidinoacetic acid, while the control group received an equivalent quantity of dietary fiber in the form of a placebo. Participants were instructed to consume the intervention once daily during breakfast, blending the experimental powder (creatine plus guanidinoacetic acid) or control powder (placebo) with 250 mL of lukewarm water and consuming it immediately afterward. Both interventions shared similar appearance, texture, and sensory characteristics.

The intervention period lasted 12 months, during which participants were advised to refrain from using any other dietary supplements. Telomere length and brain creatine levels was assessed at the baseline and at 12-month follow-up. Telomere length was assessed using the quantitative polymerase chain reaction method to measure telomere length relative to standard reference DNA, following detailed procedures described elsewhere (Cawthon, R. M., Nucleic Acids Res, 2002, 30, e47). Brain creatine levels at brain frontal grey matter was assessed using 1.5 T proton magnetic resonance, following detailed procedures described elsewhere (Ostojic, S. M., Nutrition, 2017, 33, 149-156).

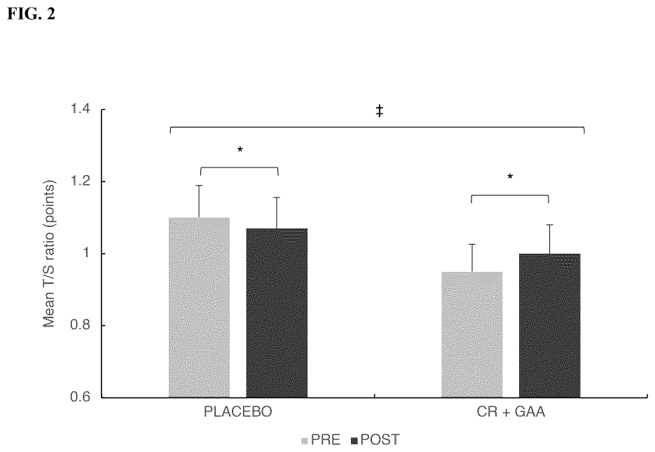

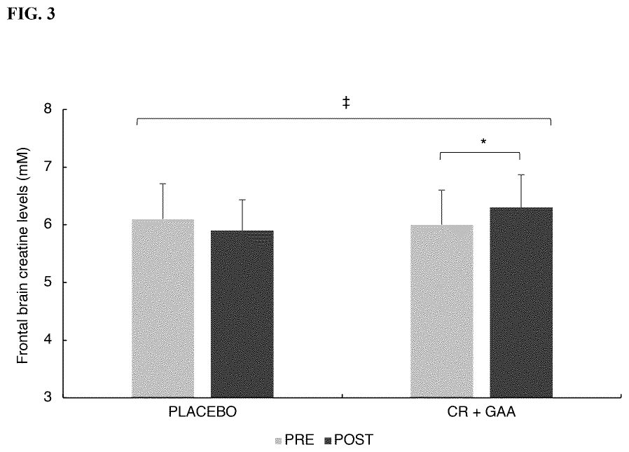

At the 12-month mark, the total number of participants randomized, who received the intended treatment, and were analyzed for the primary outcome was 16, comprising 10 in the experimental group and 6 in the control group. Changes in telomere length after intervention with 1.5 grams of creatine and 1.5 grams of guanidinoacetic acid per day for 12 months compared to telomere length of the placebo group are depicted in FIG. 2. Changes in brain creatine levels at frontal grey matter after intervention with 1.5 grams of creatine and 1.5 grams of guanidinoacetic acid per day for 12 months compared to brain creatine levels of the placebo group are depicted in FIG. 3.

The participants receiving creatine and guanidinoacetic acid experienced an increase in telomere length at 12-month follow-up as compared to the baseline (pre-intervention) levels (P<0.05). The participants receiving placebo experienced a significant decrease in telomere length at 12-month follow-up as compared to the baseline (pre-intervention) levels (P<0.05). Two-way ANOVA with repeated measures revealed a significant difference (treatment vs. time interaction) between interventions in telomere length (P<0.05), with the creatine group superior to placebo to extend telomere length.

The participants receiving creatine and guanidinoacetic acid experienced an increase in brain creatine levels at frontal grey matter at 12-month follow-up as compared to the baseline (pre-intervention) levels (P<0.05). The participants receiving placebo experienced no change in brain creatine at 12-month follow-up as compared to the baseline (pre-intervention) levels. Two-way ANOVA with repeated measures revealed a significant difference (treatment vs. time interaction) between interventions in brain creatine at frontal grey matter (P<0.05), with the creatine group superior to placebo to improve brain creatine levels. No participants reported any side effects of creatine and guanidinoacetic acid or placebo.

### Example 3: Creatine for Telomere Length in Adults ≥65 Years Old

A study aimed to evaluate the influence of creatine supplementation on telomere length in a cohort of individuals aged 65 years and over (mean age 74.8±6.9 years). This study adopted a randomized controlled posttest-only control group experimental design. Participants eligible for the trial met particular criteria, including being ≥65 years of age, devoid of any acute or chronic diseases, and lacking a history of regular creatine supplementation.

The experimental cohort was provided with a daily dosage of 2.5 grams of creatine, whereas the control group received an identical volume of dietary fiber in the form of a placebo. Participants were instructed to ingest the intervention once a day during breakfast, by combining the experimental or control powder with 250 mL of lukewarm water and consuming it immediately thereafter. Both interventions exhibited similar visual appearance, texture, and sensory attributes.

During the 12-month intervention period, participants were instructed to abstain from the use of any additional dietary supplements. Telomere length was evaluated at the 12-month follow-up using the quantitative polymerase chain reaction (PCR) method, measuring telomere length in relation to standard reference DNA. The procedures for this evaluation were meticulously detailed in previous documentation (Cawthon, R. M., Nucleic Acids Res., 2002, 30, e47).

At the 12-month assessment, the total count of randomized participants who received the designated treatment and were analyzed for the primary outcome was 53 subjects (28 women), with 23 individuals in the experimental group and 30 in the control group.

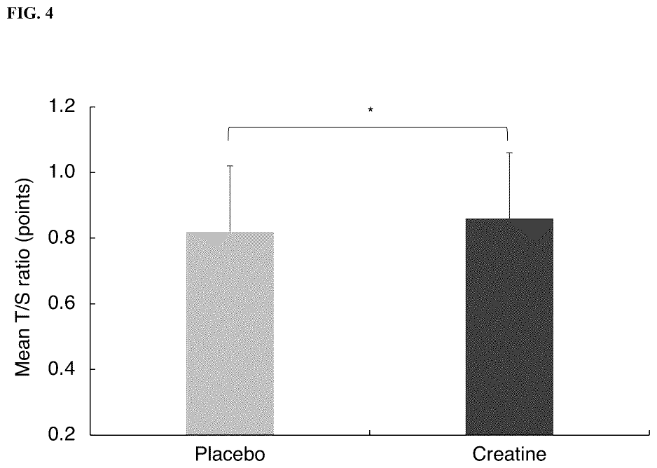

Telomere length at the 12-month follow-up is represented in FIG. 4. The figure demonstrates telomere length post-administration of 2.5 grams of creatine per day for 12 months (0.86±0.15 points) in contrast to the telomere length observed in the placebo group (0.82±0.19 points). During the trial, observations revealed higher telomere length after a 12-month administration period in participants consuming 2.5 grams of creatine per day for 12 months compared to telomere length of the placebo group (P>0.05). Telomere length was higher for 4.65% in creatine group versus placebo group. No participants reported any side effects of creatine or placebo.

1. The pharmaceutical compositions containing creatine or physiologically acceptable derivatives, salts, precursors and/or adducts thereof intended for telomere lengthening.

2. The pharmaceutical composition as claimed in claim 1, wherein insufficient telomere length results from aging, disease or environmental stress.

3. The pharmaceutical composition according to any one of claims 1 to 2, wherein the acceptable creatine derivative encompasses creatine, creatine hydrates, creatine esters phosphocreatine, guanidinoacetic acid, creatinol-O-phospate or a combination thereof.

4. The pharmaceutical composition according to any one of claims 1 to 3, wherein the acceptable creatine salts, creatine adducts, salts of physiologically acceptable creatine derivatives, and adducts of these derivatives ideally encompass acetates, aconitates, aminobutyrates, ascorbates, aspartates, chlorides, citrates, decanoates, fumarates, gluconates, hemi-sulfates, hydrochlorides isocitrates, ketoglutarates, ketoisocaproate, malates, maleates, nitrates, orotate, oxalates, oxaloacetates, phosphates, pyruvates, ribosides, succinates, sulfates, tartrates, taurinates, tetrahydrates, sodium, potassium, calcium, magnesium salts, guanidinoacetic acid adducts, or a combination thereof.

5. The pharmaceutical composition of any one of claims 1 to 4, wherein the daily dose of creatine in the composition is in the range of 1 gram to 4 grams.

6. Use of a composition comprising creatine or physiologically acceptable derivatives and/or salts, precursors and or adducts thereof as dietary supplement or as a supplement for preparation of a diet supporting telomere length.

7. The use of claim 6, wherein insufficient telomere length is caused by aging or environmental stress (smoking).

8. The use of any one of claims 6 to 7, wherein the daily dose of creatine in the composition is in the range of 1 gram to 4 grams.
